(19) United States
(12) Patent Application Publication
(10) Pub. No.: US 2024/0115987 A1
(43) Pub. Date: Apr. 11, 2024

(54) ENHANCED CO2 ADSORPTION USING TRANSITION METALS SUCH AS RU AND NI AND THEIR OXIDES IN COMBINATION WITH ALKALINE METAL OXIDES AND HIGH SURFACE AREA CARRIERS

(71) Applicant: THE TRUSTEES OF COLUMBIA UNIVERSITY IN THE CITY OF NEW YORK, New York, NY (US)

(72) Inventors: Chae JEONG-POTTER, Valley Stream, NY (US); Martha ARELLANO-TREVINO, Denver, CO (US); Robert J. FARRAUTO, Princeton, NJ (US)

(73) Assignee: THE TRUSTEES OF COLUMBIA UNIVERSITY IN THE CITY OF NEW YORK, New York, NY (US)

(21) Appl. No.: 18/526,068

(22) Filed: Dec. 1, 2023

Related U.S. Application Data
(63) Continuation of application No. PCT/US2022/031703, filed on Jun. 1, 2022.
(60) Provisional application No. 63/195,399, filed on Jun. 1, 2021.
(60) Provisional application No. 63/197,708, filed on Jun. 7, 2021.
(60) Provisional application No. 63/299,826, filed on Jan. 14, 2022.
(60) Provisional application No. 63/346,028, filed on May 26, 2022.

(51) Int. Cl. B01D 53/04; B01J 20/04; B01J 20/30; B01J 23/46; B01J 37/08; C07C 1/12

(52) U.S. Cl. CPC B01D 53/04; B01J 20/041; B01J 20/3078; B01J 23/462; B01J 37/08; C07C 1/12; B01D 2253/1124; B01D 2255/1026; B01D 2257/504

(57) ABSTRACT

A dual function material is provided that captures carbon dioxide from ambient air, i.e., direct air capture, and converts the CO₂ to a desired product such as methane. The material includes a high surface area carrier such as Al₂O₃ upon which catalysts and alkaline adsorbents are positioned proximate each other. In the presence of reactive gas such as hydrogen, the catalysts reduce the adjacent adsorbents to generate additional active sites and enhance the amount of CO₂ captured by the material. Once the material becomes saturated with CO₂, hydrogen is reintroduced to reduce the catalyst, such as ruthenium, at which time the adsorbed CO₂ can migrate from the adsorbent to the catalyst for catalytic conversion to methane. The materials can be employed in isothermal, cyclic reactor systems where target species are bound and then desorbed to reactivate the material, e.g., bind more target species for desorption and/or conversion to additional product.

### CROSS REFERENCE TO RELATED APPLICATION(S)

This application is a continuation of International Patent Application No. PCT/US2022/031703, filed Jun. 1, 2022, which claims the benefit of U.S. Provisional Application Nos. 63/195,399 filed Jun. 1, 2021, 63/197,708, filed Jun. 7, 2021, 63/299,826 filed Jan. 14, 2022, and 63/346,028, filed May 26, 2022, which are incorporated by reference as if disclosed herein in their entirety.

### STATEMENT REGARDING FEDERALLY SPONSORED RESEARCH AND DEVELOPMENT

This invention was made with U.S. Government support under DE-SC0020795 awarded by the Department of Energy. The United States Government has certain rights in the invention.

### BACKGROUND

It is widely accepted in the scientific community that the growth of anthropogenic CO₂ emissions and increasing atmospheric CO₂ concentrations need to be curtailed to reduce the impact on climate change and the associated environmental crises. In this regard, carbon capture, utilization, and storage (CCUS) technologies are becoming increasingly important. Global CO₂ emissions continue to rise with global energy demand, with energy-related emissions reaching a historical high of 33.1 Gt CO₂ in 2018 and 2019. As a result, atmospheric CO₂ concentrations have increased at an alarming average rate of 2.4 ppm/year in the past decade, surpassing 414 ppm in May 2019. High levels of atmospheric CO₂ are associated with global climate change and a myriad of other environmental crises, thereby requiring efforts to limit its growth by reducing emissions. The Intergovernmental Panel on Climate Change (IPCC) predicts that to limit warming to 1.5° C. above pre-industrial levels, CO₂ emissions will need to reach net zero by 2050. It is, however, not possible to remove all CO₂ emissions; some sectors are more difficult to decarbonize, not all emissions sources can be fit with CO₂ capture technology, and capture technologies do not capture 100% of emissions. Negative emission technologies, such as direct air capture (DAC) of CO₂, will be necessary to achieve net zero emissions and should be considered for climate change mitigation.

DAC is a process in which CO₂ is separated from the other components of ambient air and optionally concentrated for further storage or utilization. Two major separation media have gained notable traction: aqueous alkaline solutions and solid sorbents. Aqueous alkaline solutions, e.g., Ca(OH)₂, NaOH, etc., absorb CO₂ from air upon contact at ambient conditions. Release of CO₂ from aqueous alkaline solutions involves decomposition or calcination of alkaline carbonates, e.g., CaCO₃, at high temperatures (above 800° C.). Solid sorbents, e.g., metal organic frameworks (MOFs), alkali/alkaline earth metal oxides/carbonates, zeolites, etc., operate through an adsorption process in which the CO₂ is physi- or chemisorbed onto the sorbent surface. Desorption of physi- or weakly chemisorbed CO₂ from solid sorbents can be achieved at lower temperatures (around 150-250° C.). Once desorbed from either medium, the CO₂ is available for storage or utilization. Although sequestration and storage would be preferred to achieve negative emissions, utilization to a saleable and useful product is an economic incentive to further develop DAC technologies and can be a viable short-term option as storage technologies are further investigated.

Utilization of CO₂ in the synthesis of fuels by reacting with renewable H₂ from water electrolysis appears to be especially prevalent as a “Power-to-X” and hydrogen storage scheme. Given that DAC technologies have freedom of geography, they can be aptly placed near H₂ generation or waste facilities. This would remove the economic and energy burdens related to compression and long-distance transportation of the purified CO₂. A number of companies exist that have coupled DAC with fuel synthesis. Climeworks (Switzerland), which uses a solid sorbent, has partnered with other companies to establish production of synthetic fuels using CO₂ captured from the air. Carbon Engineering (Canada), which uses an alkaline aqueous solution, also aims to achieve synthetic liquid fuel production by way of their DAC technology.

Of the hydrocarbon fuels that can be produced, methane, or renewable natural gas (RNG), is quite attractive. In the presence of well-established catalysts, the Sabatier reaction, see reaction 1 below, can occur at moderate temperatures (T≤400° C.) and atmospheric pressure. High conversions of CO₂ and 100% CH₄ selectivity can be achieved with the right choice of catalysts and process conditions, generating a product stream that can be dried to achieve methane at high purities. The resulting RNG can be transported easily using the existing natural gas infrastructure.

CO₂+4H₂→CH₄+2H₂OΔH^{O}ᵣ=−165 kJ/mol (1)

For such reasons, combining CO₂ capture and methanation has gained interest in the literature. Of particular note, a way of combining DAC with CO₂ methanation (DACM) using a single reactor and material has been proposed. This process employs a supported sorbent (K₂CO₃/Al₂O₃), which adsorbs CO₂ at room temperature. In the same reactor, Ru/Al₂O₃ is used as the methanation catalyst. Upon heating to 300-350° C. and addition of H₂, the CO₂ is desorbed from the sorbent and consequently methanated over the catalyst.

Dual function materials (DFMs) have been widely investigated for a combined CO₂ capture and catalytic conversion process. DFMs comprise a sorbent and a catalytic component, dispersed on the same high surface area carrier. This combined material has the ability to operate isothermally at flue gas temperatures (300-320° C.) for both capture and conversion in a single reactor thereby, reducing energy, CO₂ compression, and transportation costs. The DFMs also enable capture of CO₂ from ambient air, i.e., DAC, with subsequent conversion to methane.

With exposure to a CO₂-including gas stream, the sorbent component allows for selective capture of CO₂; subsequently, with the introduction of H₂ (or other reactive gases such as C₂H₄), the adsorbed CO₂ is converted to a useful product (such as methane), aided by the presence of a catalyst. The sorbents most commonly studied include alkaline metal oxides such as CaO, Na₂O, and BaO. Ni, Ru, and Rh have been extensively studied as the catalytic component for methanation pathways; other catalysts, e.g., Fe, Co and Cu, have been suggested for alternative reaction pathways.

The dual functionality of these so-called “catalytic sorbents” has been extensively discussed and accepted; with the addition (or doping) of a catalyst, the sorbent is not only able to capture CO₂ but also facilitate its conversion. In fact, in some instances, the combination of sorbent and catalyst enables better conversion than the catalyst alone. In addition, the co-dispersion of the catalyst and sorbent on the same carrier enables more efficient conversion following capture.

It has been demonstrated that supported carbonates can be completely decomposed thermally at much lower temperatures than the bulk materials. By way of example, bulk Na₂CO₃ decomposes at temperatures above 800° C. while 20% Na₂CO₃/Al₂O₃ has been shown to begin decomposing at 135° C. and achieve complete decomposition between 400 and 500° C. This would suggest that after calcination at 400° C., Na₂CO₃ precursor salt impregnated onto Al₂O₃ would be fully decomposed to Na₂O adsorption sites. However, calcination at 400° C. has been shown to be insufficient to decompose all Na₂CO₃ precursor salt to Na₂O, meaning that residual carbonates remain on the DFMs and are consequently methanated.

What is desired, therefore, is a DFM that can be produced at lower temperatures yet achieve the adsorptive capacity of materials produced by high-temperature thermal decomposition, and further can catalyze the formation of target chemical products at its surface, the desorption of which helps regenerate the DFM to produce yet more product.

### SUMMARY

Aspects of the present disclosure are directed to a system for capturing carbon dioxide and converting it to a desired product. In some embodiments, the system includes at least one reactor. In some embodiments, the reactor includes one or more inlets. In some embodiments, the system includes a stream of target gas in fluid communication with one of the one or more inlets, the gas including an oxygen component and a carbon dioxide component. In some embodiments, the system includes a stream of desorptive gas in fluid communication with one of the one or more inlets. In some embodiments, the system includes a dual function material positioned within the reactor. In some embodiments, the dual function material includes a carrier portion; adsorbent portions positioned on the carrier portion, the adsorbent portions including adsorbent materials that adsorb the carbon dioxide component until the adsorbent portions are substantially saturated with carbon dioxide and desorb the carbon dioxide when exposed to the stream of desorptive gas, and catalyst portions catalyzing the formation of methane from carbon dioxide desorbed from the adsorbent portions and a reactive gas, the catalyst portions being positioned on the carrier portion adjacent the adsorbent portions.

In some embodiments, the dual function material includes less than about 0.1% by weight catalyst and between about 6% and about 10% by weight adsorbent. In some embodiments, the temperature of the dual function material is maintained at about ambient temperature during capture of the carbon dioxide component. In some embodiments, the stream of target gas is a stream of air, process effluent, or combinations thereof. In some embodiments, the carrier portion includes Al₂O₃, SiO₂, CeO₂, a zeolite, La₂O₃, TiO₂, ZnO, Nb₂O₅, ZrO₂, or combinations thereof. In some embodiments, the adsorbent portion includes Na₂O, CaO, K₂O, MgO, Li₂O, Cs₂O, Rb₂O, SrO, La₂O₃, CeO₂ or combinations thereof. In some embodiments, the catalyst portion includes one or more transition metals, transition metal compounds, or combinations thereof. In some embodiments, the catalyst portion includes one or more transition metal oxides. In some embodiments, the catalyst portion includes Ru, Cu, Mn, Ni, Cr, Fe, Mo, V, Ag, Rh, Pt, Pd, In, or combinations thereof. In some embodiments, the dual function material includes less than about 0.1% by weight Ru and between about 6% and about 10% by weight Na₂O, wherein the carrier portion includes Al₂O₃.

In some embodiments, the stream of desorptive gas includes a stream of reactive gas, the stream of reactive gas including at least 15% by weight hydrogen. In some embodiments, at least one reactor includes one or more outlets in fluid communication with an outlet stream including one or more products evolved at the dual function material, wherein the one or more products includes methane.

Aspects of the present disclosure are directed to a method of making a dual function material. In some embodiments, the method includes providing a carrier portion; loading the carrier portion with between about 6% and about 10% by weight alkaline salt adsorbent to produce an alkalinated carrier; calcining the alkalinated carrier; loading the alkalinated carrier with one or more catalysts; and contacting the alkalinated carrier with a stream of reactive gas to decompose, e.g., reduce, alkaline salt adsorbent by the one or more catalysts. Without wishing to be bound by theory, the presence of a catalyst will further reduce the adsorbent, e.g., alkaline metal precursor salts, more completely at lower temperatures producing more active sites for adsorption than just from thermal decomposition or calcination. In some embodiments, the dual function material includes less than about 0.1% by weight catalyst and between about 6% and about 10% by weight adsorbent. In some embodiments, the carrier portion includes Al₂O₃, SiO₂, CeO₂, a zeolite, La₂O₃, TiO₂, ZnO, Nb₂O₅, ZrO₂, or combinations thereof. In some embodiments, the adsorbent includes Na₂O, CaO, K₂O, MgO, Li₂O, Cs₂O, Rb₂O, SrO, La₂O₃, CeO₂ or combinations thereof. In some embodiments, the catalyst includes one or more transition metals, transition metal compounds, or combinations thereof. In some embodiments, the catalyst includes one or more transition metal oxides. In some embodiments, the catalyst includes Ru, Cu, Mn, Ni, Cr, Fe, Mo, V, Ag, Rh, Pt, Pd, In, or combinations thereof. In some embodiments, the dual function material includes less than about 0.1% by weight Ru and between about 6% and about 10% by weight Na₂O, wherein the carrier portion includes Al₂O₃.

In some embodiments, the method includes calcining the alkalinated carrier including one or more catalysts prior to contacting the alkalinated carrier with a stream of reactive gas. In some embodiments, the stream of reactive gas includes at least 15% by weight hydrogen.

Aspects of the present disclosure are directed to a method for capturing carbon dioxide and converting it to a desired product. In some embodiments, the method includes providing a dual function material. In some embodiments, the dual function material includes a carrier portion; adsorbent portions positioned on the carrier portion, the adsorbent portions including adsorbent materials that adsorb the carbon dioxide component until the adsorbent portions are substantially saturated with carbon dioxide and desorb the carbon dioxide when exposed to a stream of desorptive gas, and catalyst portions catalyzing the formation of methane from carbon dioxide desorbed from the adsorbent portions and a reactive gas, the catalyst portions being positioned on the carrier portion adjacent the adsorbent portions.

In some embodiments, the method includes contacting a stream of target gas including an oxygen component and a carbon dioxide component with the dual function material until the adsorbent portions are substantially saturated with carbon dioxide. In some embodiments, the method includes contacting a stream of desorptive gas with the dual function material that is substantially saturated with the carbon dioxide. In some embodiments, the method includes desorbing the carbon dioxide from the adsorbent portions. In some embodiments, the method includes reacting the carbon dioxide to form the desired product. In some embodiments, the temperature of the dual function material is maintained at about ambient temperature during capture of the carbon dioxide component. In some embodiments, the stream of target gas is a stream of air, process effluent, or combinations thereof. In some embodiments, the stream of desorptive gas includes at least 15% by weight hydrogen. In some embodiments, reacting the carbon dioxide to form the desired product includes reacting the carbon dioxide with the hydrogen to form methane at a temperature higher than ambient.

In some embodiments, the dual function material includes less than about 0.1% by weight catalyst and between about 6% and about 10% by weight adsorbent. In some embodiments, the carrier portion includes Al₂O₃, SiO₂, CeO₂, a zeolite, La₂O₃, TiO₂, ZnO, Nb₂O₅, ZrO₂, or combinations thereof. In some embodiments, the adsorbent portion includes Na₂O, CaO, K₂O, MgO, Li₂O, Cs₂O, Rb₂O, SrO, La₂O₃, CeO₂ or combinations thereof. In some embodiments, the catalyst portion includes one or more transition metals, transition metal compounds, or combinations thereof. In some embodiments, the catalyst portion includes one or more transition metal oxides. In some embodiments, the catalyst portion includes Ru, Cu, Mn, Ni, Cr, Fe, Mo, V, Ag, Rh, Pt, Pd, In, or combinations thereof. In some embodiments, the dual function material includes less than about 0.1% by weight Ru and between about 6% and about 10% by weight Na₂O, wherein the carrier portion includes Al₂O₃.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic representation of a dual function material (DFM) according to some embodiments of the present disclosure;

FIG. 2A is a chart of a method of making a DFM according to some embodiments of the present disclosure;

FIG. 2B is a chart of a method of making a DFM according to some embodiments of the present disclosure;

FIG. 3 is a system for capturing carbon dioxide and converting it to a desired product according to some embodiments of the present disclosure;

FIG. 4 is a chart of a method of capturing carbon dioxide and converting it to a desired product according to some embodiments of the present disclosure;

FIG. 5A is a graph showing mass gain profiles for DFMs according to some embodiments of the present disclosure upon exposure to 375 ppm CO₂, 7.5% O₂, and balance N₂ at 25° C.;

FIG. 5B is a graph showing the increase in CO₂ adsorption by DFMs according to some embodiments of the present disclosure relative to adsorbent and carrier alone;

FIG. 6 is a graph showing mass gain profiles for DFMs according to some embodiments of the present disclosure upon exposure to CO₂ relative to adsorbent and carrier alone;

FIGS. 7A-7B are graphs showing mass gain profiles for DFMs upon exposure to CO₂ relative to adsorbent and carrier alone;

FIGS. 8A-8D are graphs showing mass gain profiles for DFMs upon exposure to CO₂ relative to adsorbent and carrier alone; and

FIG. 9 is a graph showing mass gain profiles for DFMs upon exposure to CO₂ relative to adsorbent and carrier alone.

### DETAILED DESCRIPTION

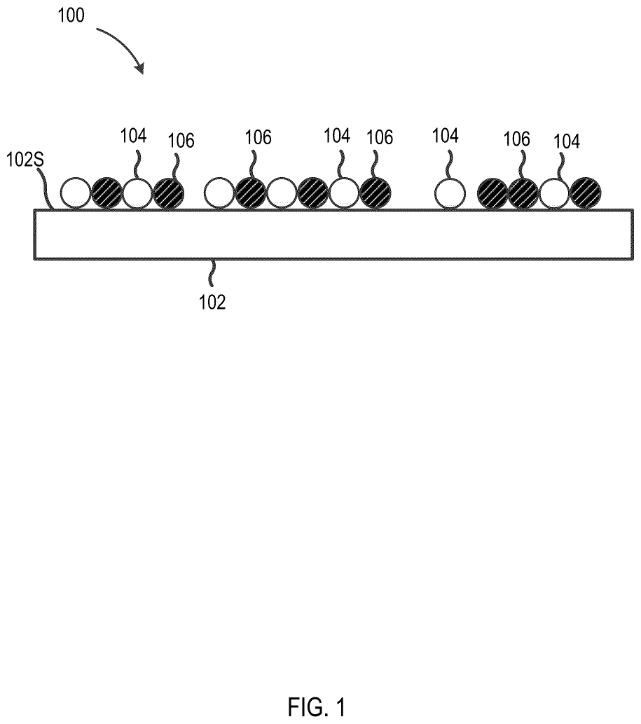

Referring now to FIG. 1, some embodiments of the present disclosure are directed to a dual function material (DFM) 100. In some embodiments, DFM 100 is configured to adsorb and desorb one or more target species. In some embodiments, upon desorption of the one or more target species, DFM 100 also facilitates conversion of the target species to one or more desired products. In some embodiments, upon desorption of the one or more target species, DFM 100 also facilitates catalytic conversion of the target species to one or more desired products. In some embodiments, the one or more target species is present at a concentration in a given medium. Upon contact of the medium with DFM 100, the one or more target species preferentially adsorbs from the medium to the DFM. In some embodiments, the medium includes one or more gases. In some embodiments, the medium includes air, an effluent stream, e.g., a waste stream from an industrial process, etc., or combinations thereof. In some embodiments, the target species is one or more gases. In some embodiments, the target species is a greenhouse gas. In some embodiments, the target species is carbon dioxide (CO₂). In some embodiments, the desired product is a renewable product intended for a reuse in a downstream process. In some embodiments, the desired product is a fuel. In some embodiments, the desired product is a hydrocarbyl material. In some embodiments, the desired product is methane.

In some embodiments, desorption of target species from DFM 100 uncovers active sites, e.g., on a surface thereof, which are again available to adsorb additional target species. In this way, DFM 100 is suitable for use in cyclic process schemes wherein target species are adsorbed thereon and thus removed from a given medium, and upon desorption of the target species, e.g., conversion of the target species to the desired product, the DFM is reactivated to adsorb yet more target species for conversion into yet more desired product, e.g., until sufficient target species is removed from the medium, until sufficient desired product has been produced, etc. In some embodiments, the capture and conversion of target species by DFM 100 is performed at a single temperature, i.e., is isothermal. Thus, processes can be designed with little to no “deadtime” for heating, cooling, or replacing of DFM 100, allowing for continuous or near-continuous generation of desired products.

In some embodiments, DFM 100 includes one or more carrier portions 102. In some embodiments, DFM 100 includes one or more adsorbent portions 104 positioned on carrier portions 102, e.g., on one or more surfaces 102S thereof. In some embodiments, carrier portions 102 are configured to reversibly immobilize adsorbent portions 104 thereon. In some embodiments, carrier portions 102 are configured to irreversibly immobilize adsorbent portions 104 thereon. In some embodiments, carrier portions 102 have a high surface area to maximize the amount of adsorbent portions 104 positioned thereon. In some embodiments, carrier portions 102 include interior as well as exterior surfaces to maximize the surface area available for binding adsorbent portions 104. As will be discussed in exemplary embodiments below, carrier portions 102 may have relatively low surface area, e.g., be composed of low surface area materials, without deviating from the embodiments of the present disclosure.

In some embodiments, carrier portions 102 are composed of any material or combination of materials that bind adsorbent portions 104 while allowing those adsorbent portions to continue to function as adsorbents, e.g., of a target species. In some embodiments, the carrier portion includes Al₂O₃, SiO₂, CeO₂, a zeolite, La₂O₃, TiO₂, ZnO, Nb₂O₅, ZrO₂, or combinations thereof.

In some embodiments, adsorbent portions 104 are composed of any material or combination of materials that reversibly adsorb a target species. In some embodiments, adsorbent portions 104 preferentially adsorb target species from a given medium, e.g., a target gas. In some embodiments, adsorbent portions 104 preferentially adsorb target species in a first environment, e.g., a target gas at a certain temperature and pressure, and desorb target species in a second environment. In some embodiments, as discussed above, the temperature in the first environment, i.e., during adsorption of the target species, is the same or substantially the same as the temperature in the second environment, i.e., during desorption of the target species. In some embodiments, the second environment is composed of one or more desorptive gases. In some embodiments, the one or more desorptive gases include one or more reactive gases. In some embodiments, the one or more desorptive gases include one or more inert gases. Thus, in an exemplary embodiment, a given medium is contacted with adsorbent portions 104, resulting in adsorption of target species from the medium to the adsorbent portions. Subsequently, one or more streams of reactive gases or inert gases are contacted with the target species-laden adsorbent portions 104, upon which the target species desorb from the adsorbent portions.

As discussed above, in some embodiments, the target species that are adsorbed by adsorbent portions 104 include one or more gases. In some embodiments, adsorbent portions 104 adsorb carbon dioxide. In some embodiments, adsorbent portions 104 adsorb target species, e.g., carbon dioxide, until the adsorbent portions are substantially saturated with the target species. In some embodiments, adsorbent portions 104 include one or more alkaline oxides, compounds including alkaline oxides, or combinations thereof. In some embodiments, adsorbent portions include Na₂O, CaO, K₂O, MgO, Li₂O, Cs₂O, Rb₂O, SrO, La₂O₃, CeO₂ or combinations thereof. In some embodiments, DFM 100 includes between about 1% and about 15% by weight adsorbent portion 104. In some embodiments, DFM 100 includes between about 2% and about 14% by weight adsorbent portion 104. In some embodiments, DFM 100 includes between about 3% and about 13% by weight adsorbent portion 104. In some embodiments, DFM 100 includes between about 4% and about 12% by weight adsorbent portion 104. In some embodiments, DFM 100 includes between about 5% and about 11% by weight adsorbent portion 104. In some embodiments, DFM 100 includes between about 5% and about 10% by weight adsorbent portion 104. In some embodiments, DFM 100 includes between about 6% and about 10% by weight adsorbent portion 104. In some embodiments, DFM 100 includes about 10% by weight adsorbent portion 104.

In some embodiments, DFM 100 includes one or more catalyst portions 106. In some embodiments, catalyst portions 106 are positioned on carrier portions 102, e.g., on one or more surfaces 102S thereof. In some embodiments, carrier portions 102 are configured to reversibly immobilize catalyst portions 106 thereon. In some embodiments, carrier portions 102 are configured to irreversibly immobilize catalyst portions 106 thereon.

In some embodiments, catalyst portions 106 are positioned on carrier portions 102 adjacent to adsorbent portions 104. In some embodiments, catalyst portions 106 are in sufficient proximity to adsorbent portions 104 so that target species desorbed from the adsorbent portions can interact with or be bound by the catalyst portions. In some embodiments, catalyst portions 106 are in sufficient proximity to adsorbent portions 104 so that target species desorbed from the adsorbent portions can interact with or be bound by the catalyst portions to catalyze conversion of the target species. In some embodiments, catalyst portions 106 are in sufficient proximity to adsorbent portions 104 so that the catalyst portions can decompose, e.g., reduce, the adsorbent portions, as will be discussed in greater detail below. In some embodiments, catalyst portions 106 are in sufficient proximity to adsorbent portions 104 so that the catalyst portions decompose the adsorbent portions in the presence of one or more inert gases. In some embodiments, catalyst portions 106 are in sufficient proximity to adsorbent portions 104 so that the catalyst portions decompose the adsorbent portions in the presence of one or more reactive gases. In some embodiments, catalyst portion 106 includes one or more transition metals, transition metal compounds, or combinations thereof. In some embodiments, catalyst portion 106 includes one or more transition metal oxides. In some embodiments, catalyst portion 106 includes Ru, Cu, Mn, Ni, Cr, Fe, Mo, V, Ag, Rh, Pt, Pd, In, or combinations thereof. In some embodiments, DFM 100 includes between about 0.01% and about 5% by weight catalyst portion 106. In some embodiments, DFM 100 includes between about 0.05% and about 4% by weight catalyst portion 106. In some embodiments, DFM 100 includes between about 0.1% and about 3% by weight catalyst portion 106. In some embodiments, DFM 100 includes less than about 0.1% by weight catalyst portion 106.

In some embodiments, catalyst portions 106 catalyze the conversion of target species adsorbed from a given medium to a desired product, e.g., as discussed above, the conversion of CO₂ to methane. In some embodiments, catalyst portions 106 catalyze the reaction of desorbed target species with a reactive gas to form the desired product. As used herein, the reactive gas is any suitable reactant or combination of reactants that can react with the desorbed target species and produce the desired product. In some embodiments, the reactive gas includes hydrogen, a hydrocarbyl compound, e.g., C₂H₄, or combinations thereof. In some embodiments, the reactive gas includes at least 15% by weight hydrogen. In some embodiments, catalyst portions 106 catalyze the formation of methane from carbon dioxide desorbed from adsorbent portions 104 and hydrogen. In some embodiments, catalyst portions 106 catalyze the formation of methane from carbon dioxide desorbed from adsorbent portions 104 and a stream of reactive gas including at least 15% by weight hydrogen. In some embodiments, DFM 100 includes Al₂O₃ as carrier portion 102, less than about 0.1% by weight Ru as catalyst portion 106, and between about 6% and about 10% by weight Na₂O as adsorbent portion 104.

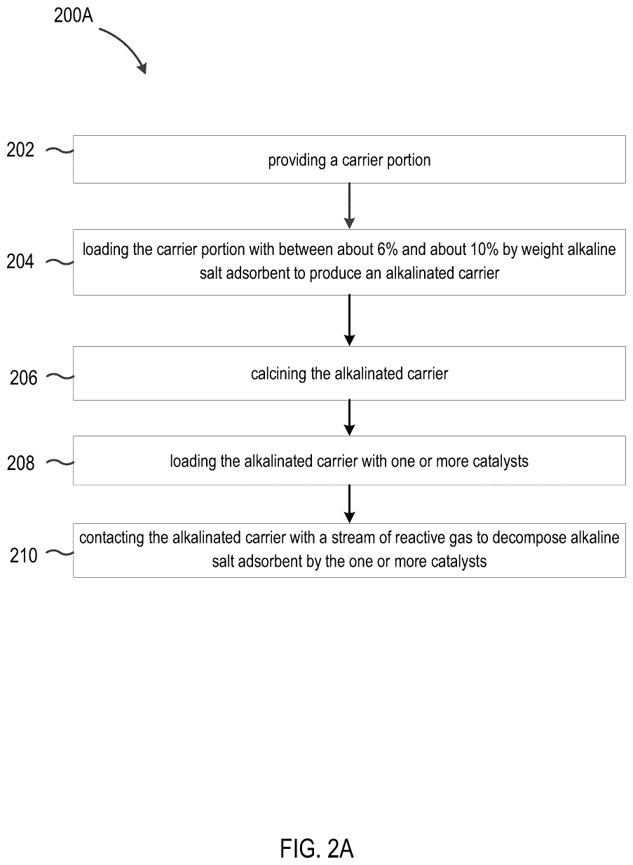

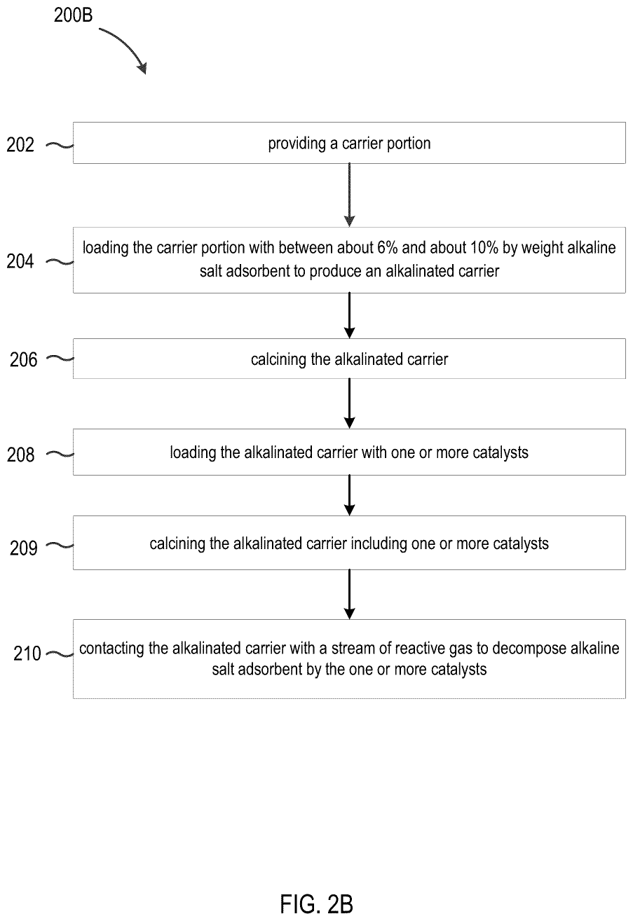

Referring now to FIG. 2A, some embodiments of the present disclosure are directed to a method 200A of making a dual function material, such as DFM 100 described above. At 202, a carrier portion is provided. As discussed above, in some embodiments, carrier portions are configured to immobilize sufficient quantities of adsorbent portions to capture desired quantities of target species from a given medium. In some embodiments, carrier portions include interior and/or exterior surfaces to maximize surface area for binding adsorbents. In some embodiments, the carrier portion includes Al₂O₃, SiO₂, CeO₂, a zeolite, La₂O₃, TiO₂, ZnO, Nb₂O₅, ZrO₂, or combinations thereof.

At 204, the carrier portion is loaded with adsorbent. In some embodiments, the adsorbent is loaded 204 on the carrier portion via any suitable process, e.g., the incipient wetness method, spray coating, etc., or combinations thereof. As discussed above, in some embodiments, the adsorbents preferentially adsorb target species in a first environment and desorb target species in a second environment. In some embodiments, the adsorbents are configured to adsorb CO₂. In some embodiments, adsorbent portions 104 include one or more alkaline oxides, compounds including alkaline oxides, or combinations thereof, thus forming an alkalinated carrier upon loading onto the carrier portion. In some embodiments, adsorbent portions include Na₂O, CaO, K₂O, MgO, Li₂O, Cs₂O, Rb₂O, SrO, La₂O₃, CeO₂ or combinations thereof. In some embodiments, the adsorbent is loaded 204 in a salt form, e.g., as alkaline carbonate, nitrate, etc., and subsequently decomposed, e.g., reduced, to an alkaline oxide such as those identified above. In exemplary embodiments, Al₂O₃ is loaded 204 with Na₂CO₃, Ca(NO₃)₂·4H₂O, Mg(NO₃)₂·6H₂O, or combinations thereof. These salts are then subsequently decomposed, e.g., reduced, to Na₂O, CaO, and MgO respectively, as will be discussed in greater detail below.

In some embodiments, at 206, the carrier loaded with adsorbent, i.e., the alkalinated carrier, is heated. In some embodiments, the carrier loaded with adsorbent is calcined. In some embodiments, heating or calcining 206 occurs in any suitable environment, e.g., stagnant air, and at any suitable temperature, e.g., above 300° C., above 400° C., etc., for any suitable duration, e.g., more than 1 hour, more than 2 hours, more than 3 hours, etc.

At 208, one or more catalysts are loaded on the carrier portion. In some embodiments, the catalyst is loaded 208 on the carrier portion via any suitable process, e.g., the incipient wetness method, spray coating, etc., or combinations thereof. As discussed above, in some embodiments, the catalyst is positioned on the carrier portion adjacent to the adsorbent. As also discussed above, the catalysts catalyze the decomposition of adsorbent, catalyze the conversion of target species adsorbed from a given medium to a desired product, e.g., aid in the conversion of CO₂ to methane, or combinations thereof. In some embodiments, the catalyst includes one or more transition metals, transition metal compounds, or combinations thereof. In some embodiments, the catalyst includes one or more transition metal oxides. In some embodiments, the catalyst includes Ru, Cu, Mn, Ni, Cr, Fe, Mo, V, Ag, Rh, Pt, Pd, In, or combinations thereof. In some embodiments, the catalyst is loaded in a salt form, e.g., as a catalytic metal nitrate compound. In exemplary embodiments, Al₂O₃ is loaded 208 with ruthenium (III) nitrosyl nitrate, nickel (II) nitrate hexahydrate, or combinations thereof.

At 210, the carrier is contacted with a stream of reactive gas to decompose the adsorbent. In some embodiments, the adsorbent is reduced during contacting 210. In some embodiments, the reactive gas includes hydrogen. In some embodiments, the stream of reactive gas includes at least 15% by weight hydrogen. In some embodiments, the adsorbent is at least partially decomposed, e.g., reduced, by the catalyst during step 210. In some embodiments, the catalyst is also at least partially reduced during step 210. Decomposition of the adsorbent salts, e.g., alkaline carbonates, produces a greater concentration of alkaline oxides on the surface of the dual function material. These oxides provide active sites for adsorption of target species such as CO₂. Reduction of the catalyst at step 210 works to further activate the catalysts. Without wishing to be bound by theory, the presence of these reduced catalysts in the dual function materials consistent with the embodiments of the present disclosure aids in the decomposition of the adsorbent salts, allowing for more extensive activation of the dual function material surface at a lower temperature. By way of example, Ru in nitrate or oxide form can be sufficiently reduced during H₂ pre-treatment to achieve catalytically active Ru⁰, thereby catalytically decomposing Na₂CO₃ to “Na₂O” and producing more active sites for CO₂ adsorption. In some embodiments, contacting 210 occurs at any suitable temperature, e.g., ambient temperature, above 100° C., above 200° C., above 300° C., above 400° C., etc., for any suitable duration, e.g., more than 1 hour, more than 2 hours, more than 3 hours, etc., to reduce adsorbent and/or catalyst for use in adsorption of one or more target species.

Referring now to FIG. 2B, some embodiments of the present disclosure include a method 200B of making a dual function material. Method 200B is consistent with method 200A discussed above, with the further inclusion of heating 209, e.g., calcining, the carrier including one or more catalysts prior to contacting 210 the carrier/adsorbent/catalyst with a stream of reactive gas. Calcining 209 can be effective to further reduce the catalyst to a more active state, allowing the catalyst to aid in the decomposition, e.g., reduction, of adsorbent and open more active sites on the dual function material for adsorption of target species. However, without wishing to be bound by theory, calcining 209 may not be suitable for use with every combination of catalyst and adsorbent. By way of example, dual function material incorporating Ru catalyst were calcined 209 to produce RuOₓ, which could then be sufficiently reduced in the presence of hydrogen to catalytically decompose Na₂CO₃ to “Na₂O” and produce more active sites. Alternatively, dual function material incorporating Ni catalyst were calcined 209 to produce NiO, which did not reduce as readily as RuO, under reduction conditions. Thus, the increase in active sites observed in the presence of calcined Ru, i.e., RuOₓ, was not as pronounced with calcined Ni, i.e., NiO. Without calcining 209, however, Ni catalyst remained in nitrate form prior to hydrogen treatment at step 210. This nitrated form was more readily reduced to Ni⁰ in the presence of hydrogen, allowing it to catalytically decompose Na₂CO₃ more completely at a lower temperature and activate more active sites on the dual function material. In view of the above, some embodiments of the present disclosure include one or more method steps to provide dual function materials with catalyst portions that are readily reduced at step 210 to increase their activity and at least partially catalytically decompose adsorbent. Therefore, embodiments of the present disclosure identify methods for making dual function materials, as well as particular methods shown to improve the performance of dual function materials made from certain combinations of carrier, adsorbent, and catalyst.

In some embodiments, as discussed above, the dual function material produced by methods 200A and/or 200B includes between about 1% and about 15% by weight adsorbent. In some embodiments, the dual function material produced by methods 200A and/or 200B includes between about 2% and about 14% by weight adsorbent. In some embodiments, the dual function material produced by methods 200A and/or 200B includes between about 3% and about 13% by weight adsorbent. In some embodiments, the dual function material produced by methods 200A and/or 200B includes between about 4% and about 12% by weight adsorbent. In some embodiments, the dual function material produced by methods 200A and/or 200B includes between about 5% and about 11% by weight adsorbent. In some embodiments, the dual function material produced by methods 200A and/or 200B includes between about 5% and about 10% by weight adsorbent. In some embodiments, the dual function material produced by methods 200A and/or 200B includes between about 6% and about 10% by weight adsorbent. In some embodiments, the dual function material produced by methods 200A and/or 200B includes about 10% by weight adsorbent. In some embodiments, the dual function material produced by methods 200A and/or 200B includes between about 0.01% and about 5% by weight catalyst. In some embodiments, the dual function material produced by methods 200A and/or 200B includes between about 0.05% and about 4% by weight catalyst. In some embodiments, the dual function material produced by methods 200A and/or 200B includes between about 0.1% and about 3% by weight catalyst. In some embodiments, the dual function material produced by methods 200A and/or 200B includes less than about 0.1% by weight catalyst. In some embodiments, the dual function material produced by methods 200A and/or 200B includes Al₂O₃ as the carrier, less than about 0.1% by weight Ru as the catalyst, and between about 6% and about 10% by weight Na₂O as the adsorbent.

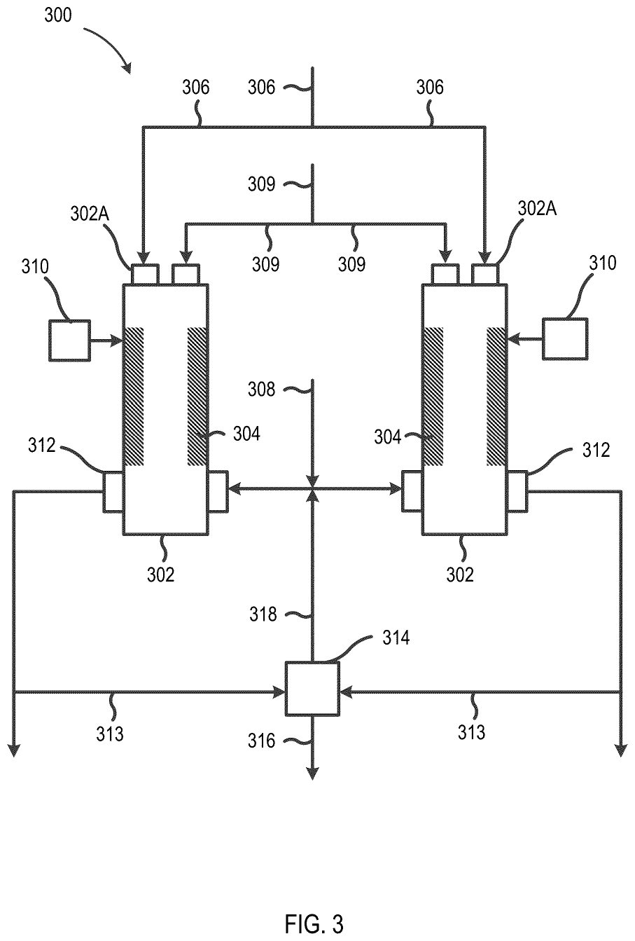

Referring now to FIG. 3, some embodiments of the present disclosure are directed to a system 300 for capturing a target species and converting it to a desired product. As discussed above, in some embodiments, the target species is one or more gases. In some embodiments, the target species is a greenhouse gas. In some embodiments, the target species is CO₂. In some embodiment, the desired product is desorbed CO₂. In some embodiments, the desired product is a renewable product intended for a reuse in a downstream process. In some embodiments, the desired product is a fuel. In some embodiments, the desired product is a hydrocarbyl material. In some embodiments, the desired product is methane.

In some embodiments, system 300 includes at least one reactor 302. In some embodiments, system 300 includes two or more reactors 302. In some embodiments, system 300 includes a plurality of reactors 302 connected in series, connected in parallel, or combinations thereof. In some embodiments, reactor 302 is any suitable size and shape to accommodate an amount of dual function material (DFM) 304 and an amount of a medium including a target species, as will be discussed in greater detail below.

In some embodiments, reactor 302 includes one or more inlets 302A. In some embodiments, one of inlets 302A is configured to deliver a first stream of gas 306 into reactor 302. In some embodiments, first stream 306 includes a target species for adsorption on DFM 304, i.e., first stream 306 includes a target gas. In some embodiments, first stream 306 includes an oxygen component. In some embodiments, the target species is CO₂, i.e., first stream 306 includes a CO₂ component. In some embodiments, first stream 306 includes a stream of air, process effluent, e.g., a waste stream from an industrial process, etc., or combinations thereof. In some embodiments, one or inlets 302A is configured to deliver a second stream of gas 308 into reactor 302. In some embodiments, second stream 308 includes one or more inert gases. In some embodiments, the inert gas includes hydrogen, nitrogen, or combinations thereof. In some embodiments, second stream 308 includes at least 15% by weight hydrogen, nitrogen, or combinations thereof. In some embodiments, one or inlets 302A is configured to deliver a third stream of gas 309 into reactor 302. In some embodiments, third stream 309 includes one or more reactive gas. In some embodiments, third stream 309 includes one or more gases configured to reduce catalyst and thus facilitate decomposition of adsorbent in DFM 304. In some embodiments, third stream 309 includes one or more gases configured to react with target species in first stream 306 to produce a desired product. In some embodiments, the reactive gas includes hydrogen, a hydrocarbyl compound, e.g., C₂H₄, or combinations thereof. In some embodiments, the reactive gas includes at least 15% by weight hydrogen. In some embodiments, second stream 308 and third stream 309 are the same stream.

Reactor 302 is configured so that when first stream 306, second stream 308, and/or third stream 309 are delivered to the reactor, those streams come into contact with DFM 304 positioned within the reactor. As discussed above, in some embodiments, DFM 304 includes a carrier portion and adsorbent portions and catalyst portions positioned on the carrier portion. In some embodiments, the adsorbent portions include adsorbent materials that adsorb target species, e.g., CO₂ component from first stream 306. In some embodiments, the adsorbent portions adsorb target species until they are substantially saturated. In some embodiments, DFM 304 is positioned and distributed in reactor 302 to maximize its surface area and thus the amount of target species that can be adsorbed before saturation is achieved. In some embodiments, the adsorbent portions are configured to desorb the target species, e.g., CO₂, when exposed to a desorptive gas. In some embodiments, the adsorptive portions are configured to desorb the target species when exposed to an inert gas, e.g., second stream 308. In some embodiments, the adsorptive portions are configured to desorb the target species when under vacuum. In some embodiments, the adsorptive portions are configured to desorb the target species when exposed to a reactive gas, e.g., third stream 309. In some embodiments, the desorbed target species, e.g., CO₂, can be removed from reactor 302. In some embodiments, the presence of the catalyst portions in DFM 304 catalyzes the formation of a desired product from the recently desorbed target species and additional reactants from the reactive gas, i.e., from third stream 309, such as hydrogen. In some embodiments, system 300 includes a heat source 310 for controlling the temperature of the interior of reactor 302, DFM 304, or combinations thereof. In some embodiments, DFM 304 is maintained at about ambient temperature during capture of target species.

In some embodiments of system 300 that include a plurality of reactors 302, continuous adsorption and desorption of target species and thus generation of desired product is enabled as first stream 306 can be fed to a first reactor (resulting in adsorption of target species in that reactor) while second stream 308/third stream 309/vacuum is applied to a second reactor (simultaneously resulting in desorption of target species in that reactor and, in some embodiments, catalytic generation of desired product). In some embodiments, once DFM 304 of the first reactor is saturated and DFM 304 of the second reactor is fully reactivated, e.g., via purging with second stream 308, first stream 306 can be fed to the second reactor and vice versa. In some embodiments, reactor 302 includes one or more outlets 312 configured to remove gaseous streams 313 from the interior of the reactor. In some embodiments, outlet 312 is in communication with one or more post-processing units 314 configured to isolate a stream of desired product 316 from stream 313. In some embodiments, post-processing unit 314 also isolates a recycle stream 318 from stream 313. In some embodiments, the recycle stream includes excess inert gas from second stream 308, excess reactive gas from third stream 309, or combinations thereof. In some embodiments, recycle stream 318 is recycled for use, e.g., in the reduction of DFM 304, desorption of target species from DFM 304, catalytic generation of desired product, etc., or combinations thereof.

In some embodiments, the carrier portion of DFM 304 includes Al₂O₃, SiO₂, CeO₂, a zeolite, La₂O₃, TiO₂, ZnO, Nb₂O₅, ZrO₂, or combinations thereof. In some embodiments, the adsorbent portion of DFM 304 includes Na₂O, CaO, K₂O, MgO, Li₂O, Cs₂O, Rb₂O, SrO, La₂O₃, CeO₂ or combinations thereof. In some embodiments, the catalyst portion of DFM 304 includes one or more transition metals, transition metal compounds, or combinations thereof. In some embodiments, the catalyst portion of DFM 304 includes one or more transition metal oxides. In some embodiments, the catalyst portion of DFM 304 includes Ru, Cu, Mn, Ni, Cr, Fe, Mo, V, Ag, Rh, Pt, Pd, In, or combinations thereof. In some embodiments, as discussed above, DFM 304 includes between about 1% and about 15% by weight adsorbent. In some embodiments, DFM 304 includes between about 2% and about 14% by weight adsorbent. In some embodiments, DFM 304 includes between about 3% and about 13% by weight adsorbent. In some embodiments, DFM 304 includes between about 4% and about 12% by weight adsorbent. In some embodiments, DFM 304 includes between about 5% and about 11% by weight adsorbent. In some embodiments, DFM 304 includes between about 5% and about 10% by weight adsorbent. In some embodiments, DFM 304 includes between about 6% and about 10% by weight adsorbent. In some embodiments, DFM 304 includes about 10% by weight adsorbent. In some embodiments, DFM 304 includes between about 0.01% and about 5% by weight catalyst. In some embodiments, DFM 304 includes between about 0.05% and about 4% by weight catalyst. In some embodiments, DFM 304 includes between about 0.1% and about 3% by weight catalyst. In some embodiments, DFM 304 includes less than about 0.1% by weight catalyst. In some embodiments, DFM 304 includes Al₂O₃ as the carrier, less than about 0.1% by weight Ru as the catalyst, and between about 6% and about 10% by weight Na₂O as the adsorbent.

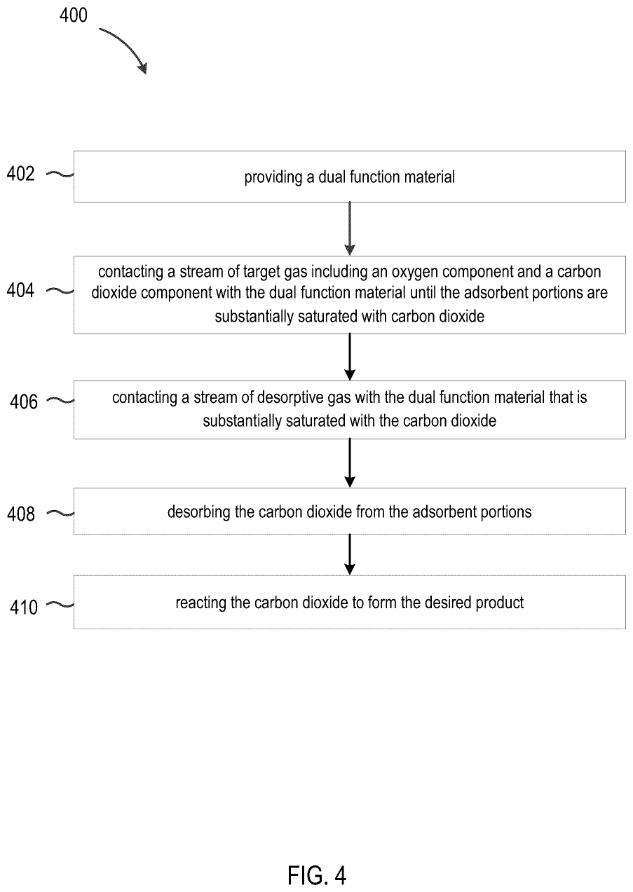

Referring now to FIG. 4, some embodiments of the present disclosure are directed to a method 400 for capturing a target species, e.g., CO₂, and converting it to a desired product, e.g., concentrated CO₂, methane, etc. At 402, a dual function material is provided. As discussed above, in some embodiments, the DFM includes a carrier portion and adsorbent portions and catalyst portions positioned on the carrier portion. In some embodiments, the carrier portion includes Al₂O₃, SiO₂, CeO₂, a zeolite, La₂O₃, TiO₂, ZnO, Nb₂O₅, ZrO₂, or combinations thereof. In some embodiments, the adsorbent portion includes Na₂O, CaO, K₂O, MgO, Li₂O, Cs₂O, Rb₂O, SrO, La₂O₃, CeO₂ or combinations thereof. In some embodiments, the catalyst portion includes one or more transition metals, transition metal compounds, or combinations thereof. In some embodiments, the catalyst portion includes one or more transition metal oxides. In some embodiments, the catalyst portion includes Ru, Cu, Mn, Ni, Cr, Fe, Mo, V, Ag, Rh, Pt, Pd, In, or combinations thereof. In some embodiments, the DFM includes between about 1% and about 15% by weight adsorbent. In some embodiments, the DFM includes between about 2% and about 14% by weight adsorbent. In some embodiments, the DFM includes between about 3% and about 13% by weight adsorbent. In some embodiments, the DFM includes between about 4% and about 12% by weight adsorbent. In some embodiments, the DFM includes between about 5% and about 11% by weight adsorbent. In some embodiments, the DFM includes between about 5% and about 10% by weight adsorbent. In some embodiments, the DFM includes between about 6% and about 10% by weight adsorbent. In some embodiments, the DFM includes about 10% by weight adsorbent. In some embodiments, the DFM includes between about 0.01% and about 5% by weight catalyst. In some embodiments, the DFM includes between about 0.05% and about 4% by weight catalyst. In some embodiments, the DFM includes between about 0.1% and about 3% by weight catalyst. In some embodiments, the DFM includes less than about 0.1% by weight catalyst. In some embodiments, the DFM includes Al₂O₃ as the carrier, less than about 0.1% by weight Ru as the catalyst, and between about 6% and about 10% by weight Na₂O as the adsorbent.

At 404, a stream of gas including the target species, e.g., target gas, is contacted with the DFM so that target species is adsorbed on the DFM. In some embodiments, the stream of gas includes including an oxygen component. In some embodiments, the stream of gas includes a CO₂ component. In some embodiments, the stream of gas is contacted 404 until the DFM is saturated or substantially saturated with the target species, e.g., CO₂ from the CO₂ component. In some embodiments, the stream of gas is a stream of air, process effluent, or combinations thereof. In some embodiments, the temperature of the DFM is maintained at about ambient temperature during capture of the target species. In some embodiments, a stream of reactive gas is also contacted with the dual function material prior to contacting 404, e.g., to further reduce the catalyst and thus the adsorbent and increase the number of active sites available to adsorb the target species.

At 406, a stream of desorptive gas is contacted with the dual function material. In some embodiments, the desorptive gas is an inert gas. In some embodiments, the desorptive gas is a reactive gas. In some embodiments, the reactive gas includes hydrogen, a hydrocarbyl compound, e.g., C₂H₄, or combinations thereof. In some embodiments, the reactive gas includes at least 15% by weight hydrogen. As discussed above, in some embodiments, the DFM is substantially saturated with the target species, e.g., CO₂. In some alternative embodiments, DFM with adsorbed target species is placed under vacuum at step 406.

At 408, the target species is desorbed from the DFM, e.g., from the adsorbent portions. In embodiments where the desired product is desorbed target species, e.g., concentrated CO2, the desorbed target species can be collected after step 408. In some embodiments, at 410, the target species is reacted to form the desired product. As discussed above, in some exemplary embodiments, adsorbed CO₂ is desorbed from the DFM in the presence of hydrogen, which is catalytically converted to methane. In some embodiments, the target species is reacted 410 at an ambient temperature. In some embodiments, the target species is reacted 410 at a temperature higher than ambient. In some embodiments, the target species evolved at step 410 can then be subsequently collected. As discussed above, upon evolution and isolation of the desired product, the dual function material is reactivated and steps of method 400 can be repeated, e.g., starting with contacting 404.

### EXAMPLES

Supported alkali and alkaline earth metal sorbents were prepared on T-Al₂O₃ (Sasol TH100 (300/160), 300 μm granules) and zirconium (IV) oxide-yttria stabilized (ZrO₂—Y) (Sigma Aldrich, submicron powder).

Four alkaline sorbents (Na₂O, CaO, MgO, and BaO) were prepared on γ-Al₂O₃ at 10% loading. Aqueous solutions of Na₂CO₃ (Sigma Aldrich), Ca(NO₃)₂·4H₂O (Sigma Aldrich), Mg(NO₃)₂·6H₂O (Sigma Aldrich), and Ba(NO₃)₂ (Sigma Aldrich) were impregnated into the carriers using the incipient wetness impregnation method. All sorbents+carrier combinations were calcined in air at 400° C.

Samples including Ru and alkaline sorbent were prepared on the two carriers as described above (γ-Al₂O₃ and ZrO₂—Y). First, the sorbents were impregnated onto the carriers as described above. After calcination, the adsorbent samples were impregnated with Ru (via a ruthenium (III) nitrosyl nitrate (Alfa Aesar) precursor salt) or Ni (as nickel (II) nitrate hexahydrate (Strem Chemicals) using the incipient wetness impregnation method. A sample of 1% Ru/Al₂O₃ (no sorbent) was also prepared on fresh Al₂O₃ granules.

Ru-containing samples were calcined in stagnant air at 250° C. and Ni-containing samples were calcined in air at 500° C. These samples, where appropriate, are designated as Ru(Ox) and Ni(Ox) where “Ox” indicates the oxide form achieved after air calcination.

Any samples in which the catalytic metal was not calcined, i.e., simply dried after impregnation onto sorbent/carrier sample, were designed as Ru(N) and Ni(N) where “N” indicates the precursor nitrate form remaining after simply drying at 120° C.

The following thermal gravimetric studies were performed in a NETZSCH TG 209 F1 Libra unit. For all studies, samples (20-30 mg) were loaded into an alumina crucible and dried in N₂ at 120° C. for 3 hours. All steps were carried out with a total gas flow of 80 m/min (STP) at 1 atm. All heating steps were carried out at 5 K/min and all cooling steps at 10 K/min unless otherwise specified. Results reported in units of mg/mgmgₛₐₘₚₗₑ were normalized using the mass of the sample obtained after pre-treatment (drying followed by hydrogen pre-reduction or calcination in air mixture); this mass represents the weight of the most active form of the DFM.

x % Ru, 10% Na₂O/Al₂O₃ (x=0.1, 0.5, 1, 3, 5) samples were studied for their room temperature CO₂ adsorption capacities. After drying, samples were reduced under a 15% H₂/N₂ stream at 300° C. for 6 hours. Once cooled to 25° C., samples were exposed to a stream of 375 ppm CO₂, 7.5% O₂, and balance N₂ for 6 hours.

x % Ru, 10% Na₂O/Al₂O₃ (x=0.1, 0.5, 1, 3, 5) samples were also tested for extent of Ru oxidation at room temperature. After drying, samples were reduced under a 15% H₂/N₂ stream at 300° C. for 6 hours. Once cooled to 25° C., samples were exposed to a stream of 7.5% O₂ in N₂ for 6 hours. These obtained mass gain values were then used to calculate the mass gain attributed to CO₂ adsorption on the samples.

Room temperature (25° C.) CO₂ adsorption capacity of the supported alkaline sorbents was studied. After drying as described above, samples were calcined in a mixture of 7:1 air/N₂ at 400° C. for 6 hours. Samples were then cooled to 25° C. in N₂ and subsequently exposed to a stream of 375 ppm CO₂, 19% O₂, and balance N₂ for 6 hours, simulating direct air capture. The test was repeated for DFM samples (catalyst+sorbent/support). After drying, samples were reduced under a 15% H₂/N₂ stream at 300° C. for 6 hours. Once cooled to 25° C., samples were exposed to a stream of 375 ppm CO₂, 19% O₂, and balance N₂ for 6 hours.

The extent of Ru oxidation on the DFMs was also studied at 25° C. After drying, samples were reduced under a 15% H₂/N₂ stream at 300° C. for 6 hours. Once cooled to 25° C., samples were exposed to a stream of 19% O₂ in N₂ for 6 hours. The obtained mass gain values were then used to calculate the mass gain attributed to CO₂ adsorption on the samples.

The CO₂ adsorption of a Ru-only sample (1% Ru/Al₂O₃) was tested at 25° C. After drying, the sample was reduced under a 15% H₂/N₂ stream at 300° C. for 6 hours. Once cooled to 25° C., the sample was exposed to a stream of 375 ppm CO₂, 19% O₂, and balance N₂ for 6 hours. A similar oxidation test was also conducted as explained in previous sections.

All fixed bed reactor studies were conducted by loading DFM 7-Al₂O₃ granules in a quartz tube reactor (O.D.=12.75 mm, I.D.=10.5 mm, L=500 mm) and secured with glass wool (Supelco Inc, USA). The remaining space in the reactor was packed with 3 mm diameter glass beads (McKesson, USA) to decrease dead volume. The reactor tube was fitted in a microthermal furnace (MTSC12.5R×18-1Z, Mellen, USA) and a K-type thermocouple (Omega, USA) at the middle of the DFM bed for temperature feedback control. Mass flow controllers were used to feed and mix compressed gases at designated flow rates. An ice bath was placed at the exit of the reactor to condense any moisture present in the system. Dry exit gas compositions were analyzed at STP using a LI-830 CO₂ gas analyzer (LI-COR, USA) for ppm-level detection of CO₂ (±2 ppm accuracy) and an Enerac 700 (Enerac, USA) for ppm-level detection of methane.

Firstly, 0.5 g of 10% Na₂O/Al₂O₃ granules were loaded in a fixed bed configuration and pre-treated at 400° C. (heating rate: 5° C./min) for 2 hours with N₂ at a total flow rate of 100 ml/min to decompose any Na₂CO₃ to “Na₂O”. The following steps were taken for subsequent adsorption/desorption cycles: CO₂ capture step at 25° C. with simulated ambient air (400 ppm CO₂/air) overnight to ensure saturation of material; heat to 400° C. in 40 minutes (9-10° C./min) in N₂; and hold at 400° C. for 80 minutes in N₂. All steps were performed at 1 atm with a total flow rate of 100 ml/min (GHSV: 12 L(STP)/h/g, 6950 h⁻¹).

Next, 0.5 g of x % Ru, 10% Na₂O/Al₂O₃ (x=0.1 and 1) granules were loaded in a fixed bed configuration and pre-treated at 300° C. (heating rate: 5° C./min) for 3 hours with 20% H₂/N₂ at a total flow rate of 16.7 ml/min (GHSV: 2L(STP)/h/g, 1160 h⁻¹) to reduce RuO, species to catalytically active Ru⁰ and to decompose any Na₂CO₃ to “Na₂O”. The following steps were taken for subsequent adsorption/methanation cycles: CO₂ capture step at 25° C. with simulated ambient air (400 ppm CO₂/air) overnight to ensure saturation of material; heat to 300° C. in 30 minutes (9-10° C./min) in 15% H₂/N₂; hold at 300° C. for 2 hours in 15% H₂/N₂. All steps were performed at 1 atm with a total flow rate of 100 ml/min (GHSV: 12 L(STP)/h/g, 6950 h⁻¹).

H₂ chemisorption tests were performed on fresh samples of x % Ru, 10% Na₂O/Al₂O₃ granules (x=0.1, 1, 3). About 0.5 g of sample was loaded into a quartz sample cell on a ChemBET Pulsar TPR/TPD unit (Quantachrome) and reduced in situ in 10% H₂/N₂ (30 ml/min, STP) at 320° C. overnight. After a 2-hour purge with N₂ (30 ml/min, STP), the temperature was decreased to 100° C. and pulses of pure H₂ were introduced. H₂ adsorbed per gram of DFM was obtained and dispersion was calculated assuming that stoichiometry for chemisorption is one H atom per Ru site.

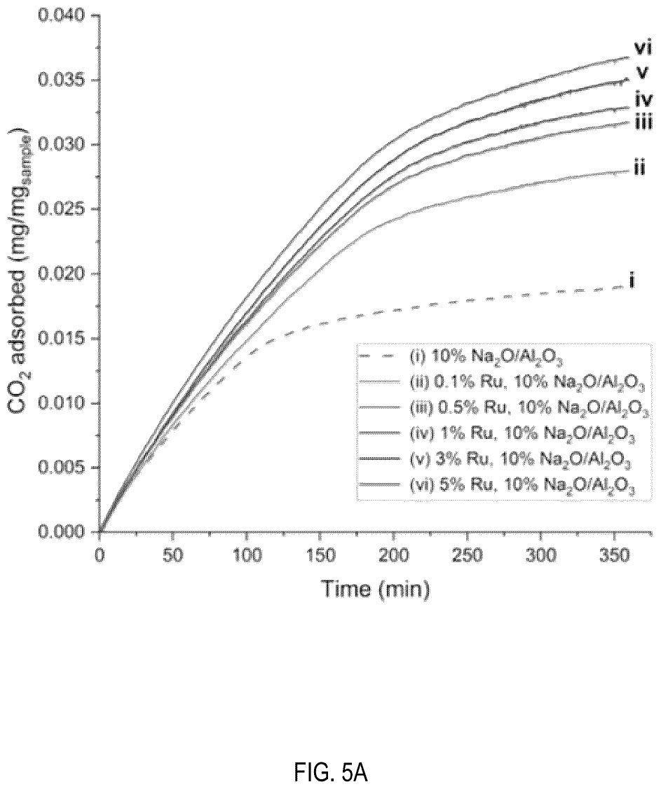

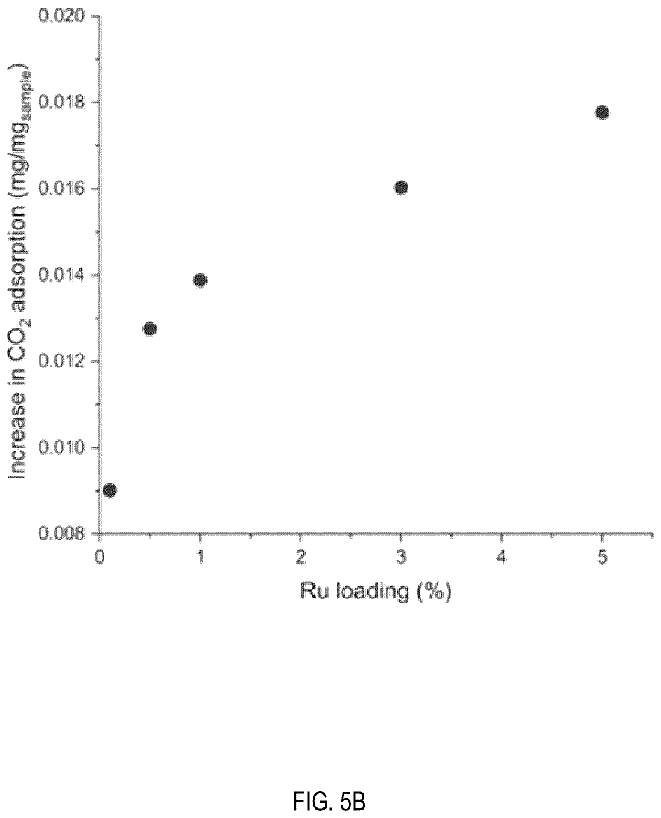

Referring now to FIGS. 5A-5B, samples of x % Ru, 10% Na₂O/Al₂O₃ (where x=0, 0.1, 0.5, 1, 3, 5) samples were exposed to a stream of 375 ppm CO₂, 7.5% O₂, and balance N₂ at 25° C. in the TGA to measure their adsorption capacities. To better account for CO₂ adsorption, the mass gain profile attributable to Ru oxidation was subtracted from the total mass gain profile of the x % Ru, 10% Na₂O/Al₂O₃ samples. All samples studied here are calcined in air, i.e., x % Ru(Ox), 10% Na₂O/Al₂O₃, before loading into the TGA for in-situ pretreatment. Ru-containing samples were pretreated with 15% H₂/N₂ at 300° C. and Na₂O/Al₂O₃ was pretreated with air at 400° C.

All of the Ru-containing samples show CO₂ capture capacities greater than that of Na₂O/Al₂O₃ alone. Even with 0.1% Ru, the CO₂ adsorption of the DFM increases by approximately 50% relative to that of the Na₂O-only sample. The CO₂ capture capacity continues to increase with higher Ru loading. This can be more clearly seen in FIG. 5B, where the increase of CO₂ adsorption relative to Na₂O/Al₂O₃ alone is shown.

The maximum CO₂ adsorbed is shown with 5% Ru at 0.0.0368 mg/mgₛₐₘₚₗₑ, equating to an overall 93.5% increase in capture capacity compared to Na₂O/Al₂O₃ only. Without wishing to be bound by theory, with higher Ru loading, there should be greater catalytic content which enhances the decomposition of the Na₂CO₃ precursor. However, the extent of enhancement is most pronounced between 0.1% and 1% Ru with the effect dampening thereafter. Further, between the 0.1% Ru and 5% Ru loading, the Ru content was increased by a factor of 50 while the adsorption capacity was improved by a factor of 2.

This is further verified by the trend in H₂ chemisorption as found in Table 1 provided below. These results show that increasing the Ru loading from 0.1% to 1% resulted in a 3.9-fold increase of H₂ adsorbed consistent with the equally dramatic increase in CO₂ adsorption shown previously. While the 0.1% Ru DFM sample adsorbed 24.5 μUL/g, the 1% Ru DFM sample adsorbed 120.7 μL/g. The increase in H₂ is not as pronounced between the 1% Ru DFM and 3% Ru DFM, which is also consistent with the dampening of the CO₂ adsorption enhancement as Ru loadings increased above 1%. It can also be seen that the dispersion decreases as the Ru loading increases, which could explain the observed dampening effect with higher Ru loadings.

TABLE 1
Results of H₂ chemisorption—specific volume of H₂ adsorbed, average
crystalline size, dispersion, and a turnover number-like quantity.

| | H₂ | Average | | “TON” |
|---|---|---|---|---|
| | adsorbed | crystalline | | [mol_{CO2-enhanced}/ |
| Sample | [μL/gₛₐₘₚₗₑ] | size [nm] | Dispersion | mol_{Ru-avail}] |
| 0.1% Ru, 10% | 24.5 | 2.02 | 22.1% | 93.7 |
| Na₂O/Al₂O₃ | | | | |
| 1% Ru, 10% | 120.7 | 4.09 | 10.9% | 29.3 |
| Na₂O/Al₂O₃ | | | | |
| 3% Ru, 10% | 127.8 | 11.6 | 3.84% | 31.9 |
| Na₂O/Al₂O₃ | | | | |

A turnover number-like (TON) quantity can be calculated using the dispersion values obtained from H₂ chemisorption to further emphasize that the enhancement effect is more efficient at lower levels of Ru. By dividing the amount of enhanced CO₂ adsorption by the amount of available Ru sites as indicated by the dispersion (mol_{CO2-enhanced}/mol_{Ru-avail}), it can be seen that the “TON” of 0.1% Ru DFM is 93.7 while it is much lower for 3% Ru DFM at 31.9.

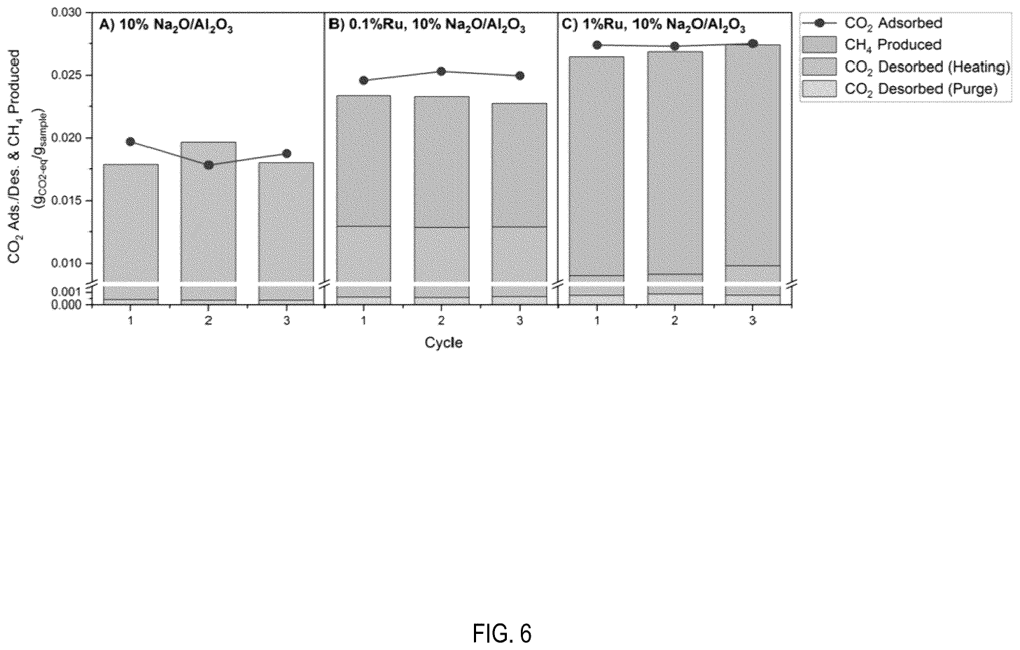

Referring now to FIG. 6, the CO₂ adsorption enhancement effect translates to increased methane production. This was verified with packed bed studies on 10% Na₂O/Al₂O₃, 0.1% Ru, 10% Na₂O/Al₂O₃, and 1% Ru, 10% Na₂O/Al₂O₃. It should be noted that the samples were pretreated in-situ with similar conditions as in the TGA experiment above.

For three cycles, 10% Na₂O/Al₂O₃ exhibited an average adsorption capacity of 0.019 g/gₛₐₘₚₗₑ. The adsorption enhancement is once again evident with the addition of Ru. The 0.1% Ru, 10% Na₂O/Al₂O₃ and 1% Ru, 10% Na₂O/Al₂O₃ samples display three-cycles of adsorption averages of 0.025 g/gₛₐₘₚₗₑ and 0.027 g/gₛₐₘₚₗₑ, respectively. The sample with a higher Ru loading has a greater adsorption capacity. The higher Ru loading sample also shows less CO₂ desorbed during heat up and more methane production. This is likely due to more favorable methanation kinetics provided by the higher Ru loading.

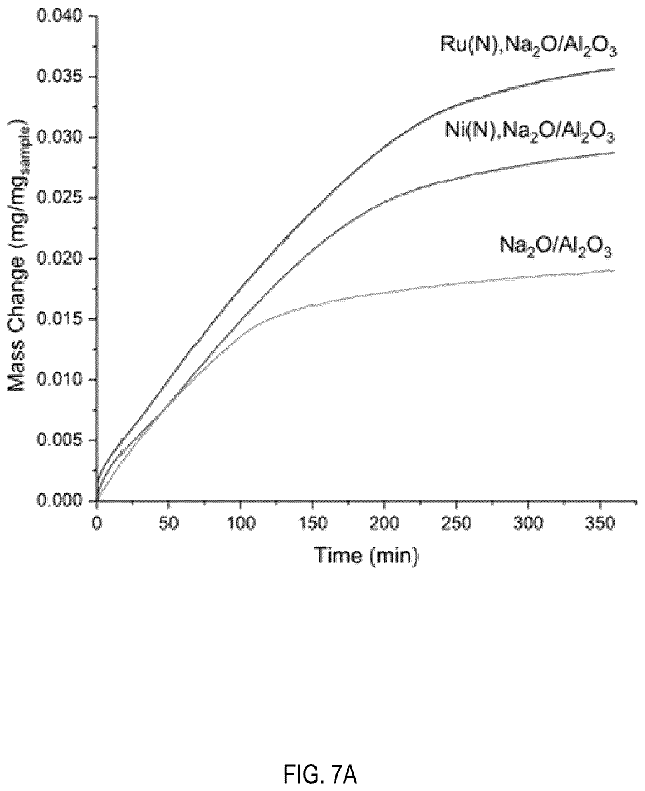

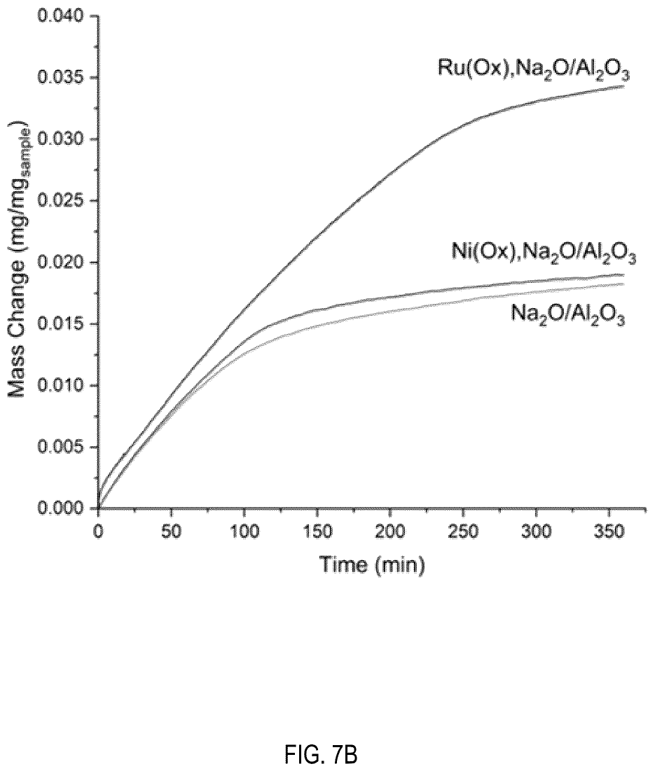

Referring now to FIG. 7A, the effect of catalytic activity on the extent of adsorption enhancement was further recognized when comparing preparation methods of Ru and Ni DFMs. Samples of catalytic metals (Ru or Ni) in combination with Na₂O/Al₂O₃ were exposed to a stream of 375 ppm CO₂, 19% O₂ and balance N₂ at room temperature (25° C.) after pre-treatment. First, samples were introduced into the instrument simply after the catalytic metal salts were impregnated onto the Na₂CO₃/Al₂O₃. This means that the catalytic metal salts were not calcined and still present in their precursor salt formed—these samples are indicated as “N” to refer to the nitrate precursors still present on the sample before in-situ pretreatment at 300° C. in 15% H₂/N₂. In this case, all samples contained 0.1% of catalytic metal and thus the mass gain associated with metal oxidation was considered insignificant. Both DFMs show higher adsorption capacities than the sorbent alone.

Referring now to FIG. 7B, the same experiment was repeated with samples that were calcined after impregnation. These samples are designated as “Ox” since they are oxidized during calcination in air prior to pretreatment in 15% H₂/N₂. The Ru(Ox), Na₂O/Al₂O₃ material (blue) shows similar adsorption behavior to Ru(N), Na₂O/Al₂O₃ thus indicating that the sample preparation method was inconsequential for the Ru DFM system. Without wishing to be bound by theory, Ru in nitrate or oxide form can be sufficiently reduced during in situ pre-reduction to methanation-active Ru⁰, thereby catalytically decomposing Na₂CO₃ to “Na₂O” and producing more active sites for CO₂ adsorption at a lower temperature. This demonstrates the ease for which Ru as an oxide can be reduced to catalytically active Ru.

Alternatively, Ni in the oxide form, following calcination of the precursor Ni salt, does not readily reduce in the presence of dilute H₂ (15%) at 300° C. and therefore, does not catalytically decompose the carbonate. Therefore, in FIG. 7B no enhancement in CO₂ adsorption is observed. In contrast, Ni in the nitrate form (“N”, solid green line) can be reduced more readily in the presence of H₂ during the pre-treatment, forming sufficient Ni⁰ available to catalytically decompose Na₂CO₃ more completely at a lower temperature than thermally.

Without wishing to be bound by theory, these results provide insights into the mechanism in which CO₂ enhancement is achieved. Sufficient catalytic activity to catalyze the decomposition of the Na₂CO₃/Al₂O₃ at a lower temperature than thermal decomposition provides an enhancement effect. In the case of Ru, both the nitrate precursor form and oxide form were sufficiently reduced during H₂ pre-treatment to achieve catalytically active Ru⁰ and as a result, both samples showed enhanced CO₂ adsorption. Nitrate reduction is possible at 300° C. to achieve catalytically active Ni; however, NiO (formed after calcination) could not be reduced at 300° C. so no Ni⁰ was available for catalytic decomposition of Na₂CO₃/Al₂O₃. As a result, the enhancement effect was observed for Ni(N) but not for Ni(Ox). Additionally, without wishing to be bound by theory, the difference in the degree of enhancement observed between Ru(N) and Ni(N) samples can be explained by the higher catalytic activity of Ru towards carbonate decomposition.

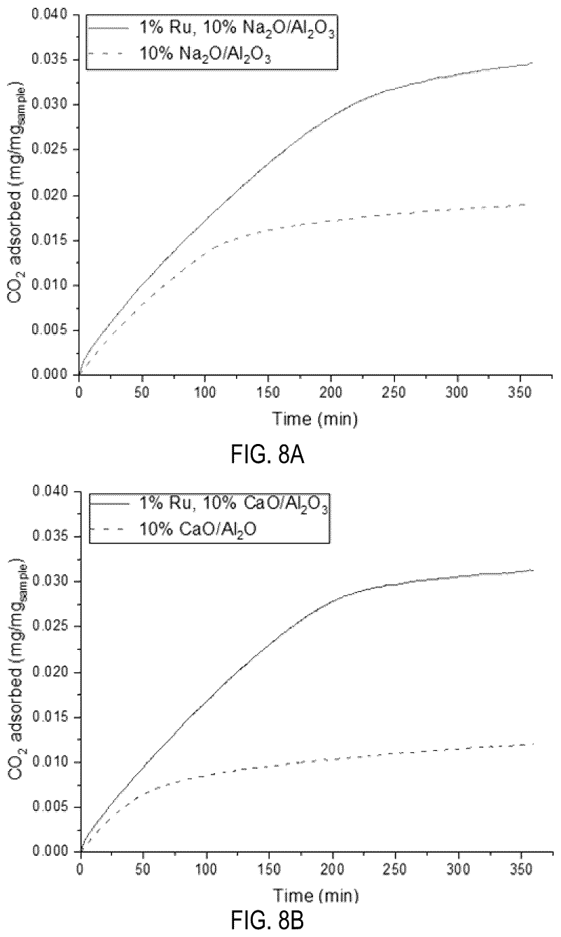

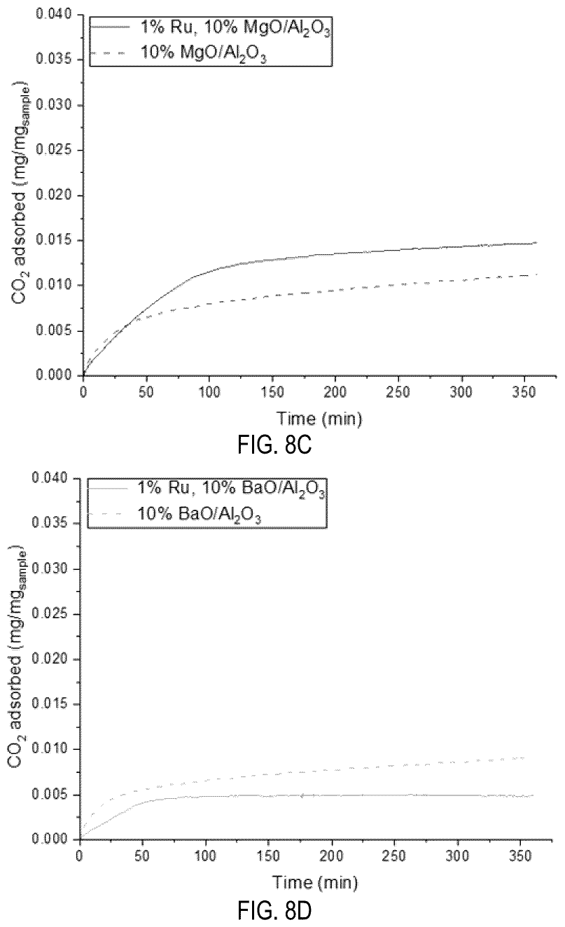

Referring now to FIGS. 8A-8D, to determine if the enhancement effect can be observed with other alkaline metal oxide sorbents, these materials supported on γ-Al₂O₃ (10% Na₂O/Al₂O₃, 10% CaO/Al₂O₃, 10% MgO/Al₂O₃, and 10% BaO/Al₂O₃) and their 1% Ru-containing counterparts were exposed to a stream of 375 ppm CO₂, 19% O₂ and balance N₂ at room temperature (25° C.). All samples were calcined in air after the final impregnation step, i.e., Ru(Ox)+sorbent/Al₂O₃, and pretreated in-situ at 300° C. with 15% H₂/N₂. The extent of oxidation was measured for all Ru-containing samples upon exposure to 19% O₂/N₂ (no CO₂). To better account for CO₂ adsorption, the mass gain profile attributable to Ru oxidation was subtracted from the total mass gain profile of the 1% Ru, 10% sorbent/Al₂O₃. Before all experiments, the samples were pretreated in-situ: the Ru-containing samples in 15% H₂/N₂ at 300° C. and the sorbent-only samples in air at 400° C.

10% Na₂O/Al₂O₃ exhibits the highest CO₂ capture capacity (in the presence of O₂) as indicated by the total mass gain. 10% CaO/Al₂O₃ and 10% MgO/Al₂O₃ exhibit similar adsorption capacities, but lower than that of 10% Na₂O/Al₂O₃. The lowest adsorption capacity was noted for 10% BaO/Al₂O₃.

Under the same O₂-containing adsorption conditions, almost all of the Ru-containing samples (solid lines) demonstrate greater CO₂ adsorption than the sorbent-only samples. 1% Ru, 10% Na₂O/Al₂O₃ shows the highest CO₂ capture capacity at 0.0346 mg/mgₛₐₘₚₗₑ, which is 0.0156 mg/mgₛₐₘₚₗₑ greater than the capture capacity seen with 10% Na₂O/Al₂O₃. This is representative of an 82.4% increase in capture capacity induced by the presence of Ru. The presence of Ru in combination with 10% CaO/Al₂O₃ shows the greatest enhancement in capture capacity. A mass gain of 0.0313 mg/mgₛₐₘₚₗₑ is attributable to CO₂ adsorption on 1% Ru, 10% CaO/Al₂O₃, which is 159% greater than the CO₂ adsorption exhibited by 10% CaO/Al₂O₃. The enhancement effect is less pronounced with 1% Ru, 10% MgO/Al₂O₃, which showed 32.8% increase in capture capacity relative to the bare sorbent. Effectively, no enhancement was seen with the combination of Ru and BaO/Al₂O₃. The observed increase in capacity implies this effect is greatest in the following order: CaO>Na₂O>MgO with little to no effect on BaO. Without wishing to be bound by theory, in the case of Ru+BaO DFM, it is suspected that given the greater molar ratio between Ru and BaO (compared to all other samples), Ru can be masking the BaO adsorption sites, explaining the lower adsorption seen with the Ru-containing sample.

The fact that the enhancement effect is observable to varying degrees between combinations indicate that the effect is not particular to Ru; in other words, not all combinations of Ru+sorbent/Al₂O₃ will exhibit enhanced adsorption capacities (given the same pretreatment conditions). Instead, without wishing to be bound by theory, Ru is more effective in catalytically decomposing some precursor salts than others. This demonstrates the use of preparation procedures for different sorbent precursors salts.

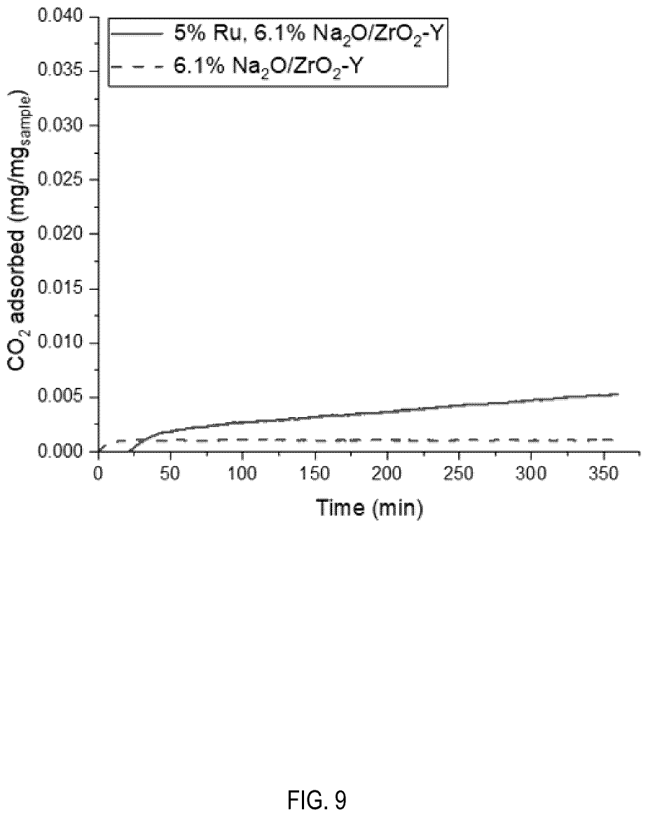

Referring now to FIG. 9, the combination of Al₂O₃ with Ru and specific alkaline sorbents was studied to determine its contribution to CO₂ adsorption enhancement. As an alternative, DFM supported on ZrO₂—Y, a support with much lower surface area, was studied. A sample of 6.1% Na₂O/ZrO₂—Y and its 5% Ru-containing counterparts were exposed to 375 ppm CO₂, 19% O₂, and balance N₂ at 25° C. Once again, the mass gain profile associated with Ru oxidation has been subtracted and all samples were calcined, i.e., Ru(Ox), Na₂O/carrier.

The oxidized Ru-containing samples demonstrate a greater mass gain in the presence of CO₂ compared to the Na₂O/ZrO₂—Y-only sample. Unlike Al₂O₃-supported Na₂O, ZrO₂—Y-supported Na₂O shows insignificant CO₂ adsorption of 0.00110 mg/mgₛₐₘₚₗₑ. This low CO₂ adsorption capacity is enhanced with the addition of 5% Ru. The total O₂-containing CO₂ adsorption on 5% Ru, 6.1% Na₂O/ZrO₂—Y is 0.00573 mg/mgₛₐₘₚₗₑ, equating to a capture capacity that is 3.6 times greater than Na₂O/ZrO₂—Y alone. This may be due to low dispersion of the Na₂CO₃ precursor, given the low surface area of the support. It is then possible that a small amount of the carbonates were decomposed at 400° C. Consequently, catalytical methanation of these hard-to-decompose carbonates plays a role in activating the capture sites, resulting in the significant increase in adsorption capacity with the addition of Ru.

The methods and systems of the present disclosure are advantageous to provide an enhanced DFM with the ability to selectively capture CO₂ from flue gas and ambient air. Direct air capture and methanation are achievable at isothermal conditions using a single material. Combinations of the carrier, adsorbent, and catalyst, e.g., Al₂O₃, Na₂O, and Ru, that exhibit particularly advantageous adsorptive capacity are identified as embodiments of the present disclosure. Further, embodiments of the present disclosure identify particular processes that maximize the ability of the DFMs to generate active sites on the surfaces thereof, and thus maximize the adsorption of CO₂.

The DFMs consistent with the embodiments of the present disclosure exhibit enhanced CO₂ adsorption at least due to the positioning of catalyst portions effective to help reduce adsorbent and free up additional CO₂ adsorption sites on the DFM. The DFMs are able to adsorb CO₂ from dilute streams (about 200 μmol/g_{DFM}, equivalent to 200 mmol/kg_{DFM}) and, upon introduction of H₂, selectively produce methane (about 150 μmol/gDFM or 150 mmol/kgDFM). Even after longtime exposure to high oxygen content during the capture step, the catalyst portion of the DFM is rapidly and completely reactivated for catalytic conversion of the adsorbed CO₂. Cyclic operation of the DFMs is made highly efficient by methanation of the adsorbed CO₂. In addition, the DFMs showed robust performance even in the presence of abnormally high moisture content in the adsorption step. Further, though there is a slight decrease in CO₂ adsorption (about 30%), the effect of moisture appears to be reversible. The DFMs are able to operate with full CO₂ adsorption capacity in dry conditions even after exposure to moisture, suggesting that the presence of moisture in the air will not contribute to irreversible sintering of the material.

The DFMs of the present disclosure also exhibit enhancement of CO₂ adsorption in O₂-including environments. The effect was demonstrated across a plurality of combinations of carrier, adsorbent, and catalyst, e.g., the addition of 5% Ru to Na₂O/ZrO₂—Y showed increase of CO₂ capture capacity, indicating that the enhancement effect can be observed even on supports with low internal surface area. It is also shown that the increased CO₂ capacity of the DFMs of the present disclosure was greater than the capture capacity exhibited by Ru (or RuOₓ) on Al₂O₃, indicating that there is a greater synergistic effect between the sorbents and Ru (and its oxides) to enable the enhancement effect. These results indicate the wide variety of ways to improve capture capacity and can be used to identify optimized combinations of catalytic metal, sorbent, and support for various applications, e.g., mitigating CO₂ emissions from O₂-including environments, especially from power plant flue gases and ambient air.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A system for capturing carbon dioxide and converting it to a desired product, the system comprising:
at least one reactor, the reactor including one or more inlets;
a stream of target gas in fluid communication with one of the one or more inlets, the gas including an oxygen component and a carbon dioxide component;
a stream of desorptive gas in fluid communication with one of the one or more inlets; and
a dual function material positioned within the reactor, the dual function material including:
a carrier portion;
adsorbent portions positioned on the carrier portion, the adsorbent portions including adsorbent materials that adsorb the carbon dioxide component until the adsorbent portions are substantially saturated with carbon dioxide and desorb the carbon dioxide when exposed to the stream of desorptive gas, and
catalyst portions catalyzing the formation of methane from carbon dioxide desorbed from the adsorbent portions and a reactive gas, the catalyst portions being positioned on the carrier portion adjacent the adsorbent portions;
wherein the dual function material includes less than about 0.1% by weight catalyst and between about 6% and about 10% by weight adsorbent,
wherein the temperature of the dual function material is maintained at about ambient temperature during capture of the carbon dioxide component, and
wherein the stream of target gas is a stream of air, process effluent, or combinations thereof.

2. The system according to claim 1, wherein the carrier portion includes Al₂O₃, SiO₂, CeO₂, a zeolite, La₂O₃, TiO₂, ZnO, Nb₂O₅, ZrO₂, or combinations thereof.

3. The system according to claim 1, wherein the adsorbent portion includes Na₂O, CaO, K₂O, MgO, Li₂O, Cs₂O, Rb₂O, SrO, La₂O₃, CeO₂ or combinations thereof.

4. The system according to claim 1, wherein the catalyst portion includes Ru, Cu, Mn, Ni, Cr, Fe, Mo, V, Ag, Rh, Pt, Pd, In, or combinations thereof.

5. The system according to claim 1, wherein the dual function material includes:
less than about 0.1% by weight Ru; and
between about 6% and about 10% by weight Na₂O;
wherein the carrier portion includes Al₂O₃.

6. The system according to claim 1, wherein the stream of desorptive gas includes a stream of reactive gas, the stream of reactive gas including at least 15% by weight hydrogen.

7. The system according to claim 1, wherein the at least one reactor includes one or more outlets in fluid communication with an outlet stream including one or more products evolved at the dual function material, wherein the one or more products includes methane.

8. A method of making a dual function material, comprising:
providing a carrier portion;
loading the carrier portion with between about 6% and about 10% by weight alkaline salt adsorbent to produce an alkalinated carrier;
calcining the alkalinated carrier;
loading the alkalinated carrier with one or more catalysts; and
contacting the alkalinated carrier with a stream of reactive gas to decompose alkaline salt adsorbent by the one or more catalysts,
wherein the dual function material includes less than about 0.1% by weight catalyst and between about 6% and about 10% by weight adsorbent.

9. The method according to claim 8, further comprising:
calcining the alkalinated carrier including one or more catalysts prior to contacting the alkalinated carrier with a stream of reactive gas.

10. The method according to claim 8, the stream of reactive gas including at least 15% by weight hydrogen.

11. The method according to claim 8, wherein the carrier portion includes Al₂O₃, SiO₂, CeO₂, a zeolite, La₂O₃, TiO₂, ZnO, Nb₂O₅, ZrO₂, or combinations thereof.

12. The method according to claim 8, wherein the adsorbent includes Na₂O, CaO, K₂O, MgO, Li₂O, Cs₂O, Rb₂O, SrO, La₂O₃, CeO₂ or combinations thereof.

13. The method according to claim 8, wherein the catalyst includes Ru, Cu, Mn, Ni, Cr, Fe, Mo, V, Ag, Rh, Pt, Pd, In, or combinations thereof.

14. The method according to claim 8, wherein the dual function material includes:
less than about 0.1% by weight Ru; and
between about 6% and about 10% by weight Na₂O;
wherein the carrier portion includes Al₂O₃.

15. A method for capturing carbon dioxide and converting it to a desired product, the method comprising:
providing a dual function material, the dual function material including:
a carrier portion;
adsorbent portions positioned on the carrier portion, the adsorbent portions including adsorbent materials that adsorb the carbon dioxide component until the adsorbent portions are substantially saturated with carbon dioxide and desorb the carbon dioxide when exposed to a stream of desorptive gas, and
catalyst portions catalyzing the formation of methane from carbon dioxide desorbed from the adsorbent portions and a reactive gas, the catalyst portions being positioned on the carrier portion adjacent the adsorbent portions;
contacting a stream of target gas including an oxygen component and a carbon dioxide component with the dual function material until the adsorbent portions are substantially saturated with carbon dioxide;
contacting a stream of desorptive gas with the dual function material that is substantially saturated with the carbon dioxide;
desorbing the carbon dioxide from the adsorbent portions; and
reacting the carbon dioxide to form the desired product,
wherein the dual function material includes less than about 0.1% by weight catalyst and between about 6% and about 10% by weight adsorbent,
wherein the temperature of the dual function material is maintained at about ambient temperature during capture of the carbon dioxide component, and
wherein the stream of target gas is a stream of air, process effluent, or combinations thereof.

16. The system according to claim 15, wherein the carrier portion includes Al₂O₃, SiO₂, CeO₂, a zeolite, La₂O₃, TiO₂, ZnO, Nb₂O₅, ZrO₂, or combinations thereof.

17. The system according to claim 15, wherein the adsorbent portion includes Na₂O, CaO, K₂O, MgO, Li₂O, Cs₂O, Rb₂O, SrO, La₂O₃, CeO₂ or combinations thereof.

18. The system according to claim 15, wherein the catalyst portion includes Ru, Cu, Mn, Ni, Cr, Fe, Mo, V, Ag, Rh, Pt, Pd, In, or combinations thereof.

19. The system according to claim 15, wherein the dual function material includes:
less than about 0.1% by weight Ru; and
between about 6% and about 10% by weight Na₂O;
wherein the carrier portion includes Al₂O₃.

20. The system according to claim 15, wherein the stream of desorptive gas includes at least 15% by weight hydrogen, and reacting the carbon dioxide to form the desired product further comprises:
reacting the carbon dioxide with the hydrogen to form methane at a temperature higher than ambient.
